(19) United States
(12) Patent Application Publication
(10) Pub. No.: US 2026/0216023 A1
(43) Pub. Date: Jul. 30, 2026

(54) SKINCARE FORMULATIONS AND METHODS USING SAME

(71) Applicant: Kenvue Brands LLC, Summit, NJ (US)

(72) Inventors: Simarna Kaur, Neshanic Station, NJ (US); Alexandria Dinapoli Marzano, Plainsboro, NJ (US); Jessica Maciejewski, Springfield, PA (US); Liliam A. Moreira, Lake Hiawatha, NJ (US)

(21) Appl. No.: 19/147,474

(22) PCT Filed: Jan. 11, 2024
(86) PCT No.: PCT/IB2024/050299

§ 371 Date: Jul. 11, 2025

Related U.S. Application Data
(60) Provisional application No. 63/479,382, filed on Jan. 11, 2023.

(51) Int. Cl. A61K 8/36; A61K 8/34; A61K 8/37; A61K 8/81; A61K 8/891; A61K 8/9789; A61Q 19/08

(52) U.S. Cl. CPC A61K 8/361; A61K 8/345; A61K 8/375; A61K 8/8147; A61K 8/891; A61K 8/9789; A61Q 19/08

(57) ABSTRACT

Provided are skincare compositions comprising: an emollient; a viscosity increaser; an emulsifier; a humectant; and a compound having retinol-like activity. Also provided are methods of treating the skin, the method comprising applying topically to skin the skincare compositions. Also provided are skincare compositions comprising a) dimethicone; b) a viscosity increaser selected from the group consisting of polyacrylate crosspolymer-6, polyacrylate-13, sorbitan stearate, and combinations thereof; c) an emulsifier selected from the group consisting of sorbitan olivate, ceterayl olivate, and combinations thereof; d) glycerin; and e) a polar botanical extract of Acronychia acidula.

### FIELD

The present invention generally relates to compositions suitable for use on skin and particularly compositions which activate retinoic pathways.

### BACKGROUND

The human skin is subject to certain aging processes, some of which are attributable to intrinsic processes (e.g. chronoaging) and some of which are attributable to exogenous factors (e.g. photo-aging). In addition, temporary or even lasting changes to the skin can occur, such as acne, greasy or dry skin, keratoses, rosacea, light-sensitive, inflammatory, erythematous, and allergic or autoimmune-reactive reactions, such as dermatosis and photodermatosis.

The consequences of the above-mentioned ageing processes can include thinning of the skin, weaker interlacing of epidermis and dermis, and a reduction in the number of cells and the supplying blood vessels. These consequences are often undesirable, and individuals suffering from these issues will look to topical treatments to address them.

Retinoids have been used for treating skin conditions caused by intrinsic aging, exogenous factors, acne or skin diseases. However, despite the beneficial effects of retinoid treatment, its benefits are limited due to skin irritation of retinoids. These side effects can restrict the use of retinoids, and particularly so for individuals having sensitive skin.

To date, the search for alternative compounds to replace retinoids, and particularly for individuals having sensitive skin, has produced limited success in treating skin conditions associated with aging, such as skin atrophy, acne, photo-aging, and in reducing the appearance of wrinkles, fine lines, stretch marks, or cellulite.

Accordingly, there is a need for alternatives to traditional retinoids which are efficacious but gentle enough for use on individuals having sensitive skin, as well as formulations for delivering these alternatives.

### SUMMARY

Accordingly, one aspect of the invention pertains to a skincare composition comprising:
a. an emollient;
b. a viscosity increaser;
c. an emulsifier;
d. a humectant; and
e. a compound of Formula I:

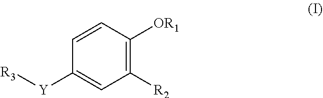

wherein:
   R₁ is selected from the group consisting of C₁-C₂₀ alkyl, C₂-C₂₀ alkenyl, C₂-C₂₀ alkynyl, and C₃-C₈ cycloalkyl or aryl;
   R₂ is selected from the group consisting of hydrogen, hydroxyl, C₁-C₆ alkyl, C₂-C₆ alkenyl, C₂-C₆ alkynyl, C₃-C₈ cycloalkyl or aryl, —OC₁-C₆ alkyl, —OC₂-C₆ alkenyl, —OC₂-C₆ alkynyl, —OC₃-C₈ cycloalkyl or aryl, thiol, —SC₁-C₆ alkyl, —SC₂-C₆ alkenyl, —SC₂-C₆ alkynyl, —SC₃-C₈ cycloalkyl or aryl, —NR₄C₁-C₆ alkyl, —NR₄C₂-C₆ alkenyl, —NR₄C₂-C₆ alkynyl, and —NR₄C₃-C₈ cycloalkyl or aryl;
   R₃ is selected from —CO₂H, —CO₂R₄ or an isosteric equivalent of a carboxy group, wherein R₄ is C₁-C₆ alkyl, C₂-C₆ alkenyl, C₃-C₈ cycloalkyl or aryl; and Y is —(CH₂—CH₂)—, —(CH═CH)—, or —(C═C)—;
      or a cosmetically acceptable salt thereof

In one or more embodiments, R₁ is selected from the group consisting of C₅-C₁₆ alkyl, C₅-C₁₆ alkenyl, and C₅-C₁₆ alkynyl; R₂ is selected from the group consisting of hydrogen, hydroxyl, —OC₁-C₆ alkyl, —OC₂-C₆ alkenyl, —OC₂-C₆ alkynyl, —OC₃-C₈ cycloalkyl; R₃ is selected from —CO₂H, —CO₂R₄ wherein R₄ is C₁-C₆ alkyl, or an isosteric equivalent of a carboxy group; and Y is —(CH₂—CH₂)— or —(CH═CH)—. In some embodiments, R₁ is selected from the group consisting of C₅-C₁₆ alkenyl; and R₂ is selected from the group consisting of hydrogen or —OC₁-C₃ alkyl. In one or more embodiments, the compound of Formula I is selected from the group consisting of 3-(4-farnesyloxyphenyl)-propionic acid, 3-(4-farnesyloxy-3-hydroxyphenyl)-propionic acid, 3-(4-farnesyloxy-3-methoxyphenyl)-propionic acid, ethyl esters thereof, and combinations of two or more thereof. In some embodiments, the compound of Formula I is comprises 3-(4-farnesyloxyphenyl)-propionic acid. In one or more embodiments, the concentration of the compound of Formula I is present in an amount ranging from about 0.0001% to about 1% by total weight of the composition. In some embodiments, the skincare composition comprises a botanical extract comprising the compound of Formula I. In one or more embodiments, the botanical extract comprises an extract of a plant of the genus Acronychia. In some embodiments, the botanical extract is an extract of Acronychia acidula. In one or more embodiments, the botanical extract is a polar extract. In some embodiments, the botanical extract is present in an amount ranging from about 0.01% to about 5% by total weight of the composition. In one or more embodiments, the extract of Acronychia acidula comprises 3-(4-farnesyloxyphenyl)-propionic acid in a concentration ranging from about 0.01% to about 0.5% by total weight of the composition. In some embodiments, the emollient is a silicone or silicone-based emollient. In one or more embodiments, the emollient is selected from the group consisting of dimethicone, dimethicone cross-polymer, phenyl trimethicone, cetyl trimethicone, amodimethicone, caprylyl methicone, and combinations thereof. In some embodiments, the emollient is present in a concentration of about 0.1% to about 15% by weight. In one or more embodiments, the viscosity increaser is a rheology modifier that provide yield to the composition and is compatible at pH less than about 5. In some embodiments, the viscosity increaser is selected from the group consisting of polyacrylate crosspolymer-6, polyacrylate-13, sorbitan stearate, polyacrylamide, C13-14 isoparaffin, laureth-7, ammonium acryloyldimethyltaurate/VP copolymer, carbomer, and combinations thereof. In one or more embodiments, the viscosity increaser is present in a concentration of about 0.1% to about 5% by weight. In some embodiments, the emulsifier comprises a liquid crystal emulsifier. In one or more embodiments, the emulsifier is selected from the group consisting of sorbitan olivate, ceterayl olivate, C12-20 alkyl glucoside, C14-22 alcohols, sorbitan stearate, sorbityl laurate, polyglyceryl-6 pentaoleate, and combinations thereof. In some embodiments, the emulsifier is present in a concentration of about 0.1% to about 5% by weight. In one or more embodiments, the humectant comprises a polar humectant. In some embodiments, the humectant is selected from the group consisting of glycerin, sorbitol, xylitol, hyaluronic acid, lactic acid, and combinations thereof. In one or more embodiments, the humectant is present in a concentration of about 0.5% to about 15% by weight.

Another aspect of the invention pertains to a method of treating the skin, the method comprising applying topically to skin any of the skincare compositions described herein.

In some embodiments, the method comprises a increasing CRABP2 expression in skin cells.

Another aspect of the invention pertains to a skincare composition comprising:
a. dimethicone;
b. a viscosity increaser selected from the group consisting of polyacrylate crosspolymer-6, polyacrylate-13, sorbitan stearate, and combinations thereof;
c. an emulsifier selected from the group consisting of sorbitan olivate, ceterayl olivate, and combinations thereof;
d. glycerin; and
e. a polar botanical extract of Acronychia acidula.

### DETAILED DESCRIPTION

It is believed that one skilled in the art can, based on the description herein, utilize the present invention to its fullest extent. The following specific embodiments are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference.

Unless otherwise indicated, percentages used to express amounts of ingredients are percentage by weight (referred to as “weight %,” “wt %”, “% by weight” or “% (W/W)”). Similarly, weight ratios used to express relative proportions of ingredients are also determined using percentage by weight (i.e., weight ratios are calculated by dividing the percentage by weight of one ingredient by another). Unless stated otherwise, all ranges are inclusive of the endpoints, e.g., “from 4 to 9” includes the endpoints 4 and 9.

As used herein, a composition that is “essentially free” of an ingredient means the composition that has about 2% or less of that ingredient by weight based on the total weight of the composition. Preferably, a composition that is essentially free of an ingredient has about 1% or less, more preferably about 0.5% or less, more preferably about 0.1% or less, more preferably about 0.05 or less, more preferably about 0.01% or less by weight based on the total weight of composition of the ingredient. In certain more preferred embodiments, a composition that is essentially free of an ingredient is free of the ingredient, i.e. has none of that ingredient in the composition.

As used herein, “cosmetically/dermatologically acceptable” means that the ingredients which the term describes are suitable for use in contact with tissues (e.g., the skin or hair) without undue toxicity, incompatibility, instability, irritation, allergic response, and the like. As will be recognized by one of skill in the art, cosmetically/dermatologically acceptable salts are acidic/anionic or basic/cationic salts.

As used herein, the term “safe and effective amount” means an amount of the extract or of the composition sufficient to induce the desired effect, but low enough to avoid serious side effects. The safe and effective amount of the compound, extract, or composition will vary with e.g. the age, health and environmental exposure of the end user, the duration and nature of the treatment, the specific extract, ingredient, or composition employed, the particular pharmaceutically-acceptable carrier utilized, and like factors.

As used herein, the term “about” refers to within 5% weight, within 4% weight, within 3% weight, within 2.5% weight, within 2% weight, or within 1% weight of a disclosed value.

In general, IUPAC nomenclature rules are used herein and according to the following term definitions.

The term “substituted,” refers to a core molecule in which one or more hydrogen atoms have been replaced with that amount of substituents allowed by available valences. Substitution is not limited to the core molecule, but may also occur on a substituent radical, whereby the radical becomes a linking group.

The term “independently selected” refers to two or more substituents that may be selected from a substituent variable group, wherein the selected substituents may be the same or different.

The term “dependently selected” refers to one or more substituent variables that are specified in an indicated combination for substitution in a core molecule (e.g. variables that refer to groups of substituents appearing in a tabular list of compounds).

Acceptable salts from inorganic bases include, for example, sodium or potassium salts, and the like. Acceptable salts from organic bases include, for example, salts formed with primary, secondary, or tertiary amines, and the like.

One aspect of the invention pertains to a skincare composition comprising:
(a) an emollient;
(b) a viscosity increaser;
(c) an emulsifier;
(d) a humectant; and
(e) a compound of Formula I:

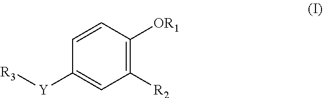

wherein:
   R₁ is selected from the group consisting of C₁-C₂₀ alkyl, C₂-C₂₀ alkenyl, C₂-C₂₀ alkynyl, and C₃-C₈ cycloalkyl or aryl;
   R₂ is selected from the group consisting of hydrogen, hydroxyl, C₁-C₆ alkyl, C₂-C₆ alkenyl, C₂-C₆ alkynyl, C₃-C₈ cycloalkyl or aryl, —OC₁-C₆ alkyl, —OC₂-C₆ alkenyl, —OC₂-C₆ alkynyl, —OC₃-C₈ cycloalkyl or aryl, thiol, —SC₁-C₆ alkyl, —SC₂-C₆ alkenyl, —SC₂-C₆ alkynyl, —SC₃-C₈ cycloalkyl or aryl, —NR₄C₁-C₆ alkyl, —NR₄C₂-C₆ alkenyl, —NR₄C₂-C₆ alkynyl, and —NR₄C₃-C₈ cycloalkyl or aryl;
   R₃ is selected from —CO₂H, —CO₂R₄ or an isosteric equivalent of a carboxy group, wherein R₄ is C₁-C₆ alkyl, C₂-C₆ alkenyl, C₃-C₈ cycloalkyl or aryl; and Y is —(CH₂—CH₂)—, —(CH═CH)—, or —(C═C)—;
      or a cosmetically acceptable salt thereof.

The compositions described herein have been surprisingly discovered to activate various biomarkers associated with retinoids, which are known for their anti-acne, anti-aging and skin barrier properties. Additionally, the formulation ingredients as a whole can enhance the delivery of the retinol-like compounds.

### Emollient

As used herein, an “emollient” refers to compounds that help to maintain the soft, smooth, and pliable appearance of the skin (e.g., by remaining on the skin surface or in the stratum corneum to act as a lubricant). Examples of emollients include those found in Chapter 35, pages 399-415 (Skin Feel Agents, by G Zocchi) in Handbook of Cosmetic Science and Technology (edited by A. Barel, M. Paye and H. Maibach, Published in 2001 by Marcel Dekker, Inc New York, N.Y.), and include, but are not limited to, esters (e.g., isopropyl palmitate), petrolatum, hexyldecyl stearate and plant, nut, and vegetable oils and butters such as Butyrospermum parkii (shea) butter, Euphorbia cerifera (candelilla) wax; hydrogenated vegetable oil, macadamia nut oil, rice bran oil, grape seed oil, palm oil, prim rose oil, hydrogenated peanut oil, and avocado oil.

In one or more embodiments, the emollient comprises a silicone or silicone-based emollient (i.e., a compound containing silicone units). In yet further embodiments, the emollients is selected from the group consisting of dimethicone, dimethicone cross-polymers, phenyl trimethicone, cetyl trimethicone, amodimethicone, caprylyl methicone, and combinations thereof.

The emollient(s) may be present in an amount of from about 0.5% to about 15%, about 1 to about 10%, about 1% to about 8%, about 1% to about 6%, about 1% to about 5%, about 1% to about 4%, about 1% to about 3%, or about 1.5% to about 3.5% by weight % of the total composition. In one or more embodiments, the emollient comprises dimethicone and is present in an amount of from about 0.5% to about 15%, about 1 to about 10%, about 1% to about 8%, about 1% to about 6%, about 1% to about 5%, about 1% to about 4%, about 1% to about 3%, or about 1.5% to about 3.5% by weight % of the total composition.

### Viscosity Increaser

As used herein, “viscosity increaser” (also known as thickeners) refers to compounds which function as rheology modifiers which provide yield to the composition. Examples of such thickening agents include: carbomers (e.g., CARBOPOL ULTREZ 30 polymer), cetyl alcohol, electrolytes (e.g. Sodium Chloride, Ammonium Chloride, Magnesium Chloride); naturally-derived polysaccharides (e.g. Xanthan Gum, Dehydroxanthan Gum, Cyamopsis Tetragonoloba (Guar) Gum, Cassia Gum, Chondrus Crispus (Carrageenan) Gum, Alginic Acid and alginate gums (Algin, Calcium Alginate, etc.), Gellan Gum, Pectin, Microcrystalline Cellulose); derivatives of natural polysaccharides (e.g. Hydroxyethylcellulose, Ethyl Hydroxyethylcellulose, Cetyl Hydroxyethylcellulose, Methylcellulose, Hydroxypropylcellulose, Sodium Carboxymethylcellulose, Hydroxypropyl Methylcellulose, Hydroxypropyl Guar, Carboxymethyl Hydroxypropyl Guar, C18-22 Hydroxylalkyl Hydroxypropyl Guar); alkali-swellable emulsion (ASE) polymers (e.g. Acrylates Copolymer, available under the trade name Carbopol® AQUA SF-1 from Noveon Consumer Specialties, Brecksville, Ohio, and Acrylates Copolymer available under the trade name Aculyn™ 33 from Dow Personal Care, Spring House, Pa.); hydrophobically-modified alkali-swellable emulsion (HASE) polymers (e.g. Acrylates/Steareth-20 Methacrylate Copolymer, Acrylates/Steareth-20 Methacrylate Crosspolymer, and Acrylates/Ceteth-20 Itaconate Copolymer); hydrophobically-modified acid-swellable emulsion polymers (e.g. Acrylates/Aminoacrylates/C10-30 Alkyl PEG-20 Itaconate Copolymer and Polyacrylate-1 Crosspolymer); hydrophobically-modified acrylate crosspolymers, such as Acrylates C10-30 Alkyl Acrylates Crosspolymer, available under the trade name Carbopol® 1382 from Lubrizol Corp., Brecksville, Ohio; sodium polyacrylate (available as COSMEDIA SP), and hydrophobic non-ethoxylated micellar thickeners (e.g. Glyceryl Oleate, Cocamide MIPA, Lauryl Lactyl Lactate, or Sorbitan Sesquicaprylate).

In one or more embodiments, the viscosity increaser is compatible at a pH of less then about 5. In one or more embodiments, the viscosity increaser is selected from the group consisting of polyacrylate crosspolymer-6, polyacrylate-13, sorbitan stearate, polyacrylamide, C13-14 isoparaffin, laureth-7, ammonium acryloyldimethyltaurate/VP copolymer, carbomer, and combinations thereof. Suitable viscosity increasers are available under tradenames Sepigel 305 (INCI-Polyacrylamide & C13-14 Isoparaffin & Laureth-7), Aristoflex AVC: (INCI-Ammonium Acryloyldimethyltaurate/VP Copolymer) and CARBOPOL Ultrez 30 (INCI-Carbomer).

The viscosity increaser(s) may be present in an amount of from about 0.1% to about 5%, about 0.2 to about 4.5%, about 0.3% to about 4%, about 0.5% to about 3.5%, about 0.5% to about 3%, about 1% to about 3%, about 1.5% to about 3%, or about 2% to about 2.5% by weight % of the total composition. In one or more embodiments, the viscosity increaser is selected from the group consisting of polyacrylate-13 and polyisobutene and polysorbate 20, polyacrylate crosspolymer-6, and combinations thereof, and is present in an amount of from about 0.1% to about 5%, about 0.2 to about 4.5%, about 0.3% to about 4%, about 0.5% to about 3.5%, about 0.5% to about 3%, about 1% to about 3%, about 1.5% to about 3%, or about 2% to about 2.5% by weight % of the total composition.

### Emulsifier

As used herein, “emulsifier” (also known as surfactants) refers to compounds which function to help form and/or stabilize an emulsion. Surfactants/emulsifiers may include anionic, cationic, nonionic and amphoteric surfactants. Suitable emulsifiers include olive-derived emulsifiers, such as olivates. For example, one suitable emulsifier includes Olivem 1000 (sold by Hallstar), which is a combination of cetearyl olivate and sorbitan olivate. Examples of suitable nonionic surfactants include, but are not limited to the fatty alcohol acid or amide ethoxylates, monoglyceride ethoxylates, sorbitan ester ethoxylates alkyl polyglycosides, and mixtures thereof. One suitable nonionic surfactant is the polyoxyethylene derivatives of polyol esters, wherein the polyoxyethylene derivative of polyol ester (1) is derived from (a) a fatty acid containing from about 8 to about 22, and preferably from about 10 to about 14 carbon atoms, and (b) a polyol selected from sorbitol, sorbitan, glucose, α-methyl glucoside, polyglucose having an average of about 1 to about 3 glucose residues per molecule, glycerin, pentaerythritol and mixtures thereof, (2) contains an average of from about 10 to about 120, and preferably about 20 to about 80 oxyethylene units; and (3) has an average of about 1 to about 3 fatty acid residues per mole of polyoxyethylene derivative of polyol ester.

Examples of polyoxyethylene derivatives of polyol esters include, but are not limited to PEG-80, sorbitan laurate, and polysorbate 20. PEG-80 sorbitan laurate, which is a sorbitan monoester of lauric acid ethoxylated with an average of about 80 moles of ethylene oxide, is available commercially from ICI Surfactants of Wilmington, Del. under the tradename, “Atlas G-4280.” Polysorbate 20, which is the laurate monoester of a mixture of sorbitol and sorbitol anhydrides condensed with approximately 20 moles of ethylene oxide, is available commercially from ICI Surfactants of Wilmington, Del. under the tradename “Tween 20.”

Another class of nonionic surfactants includes long chain alkyl glucosides or polyglucosides, which are the condensation products of (a) a long chain alcohol containing from about 6 to about 22, and preferably from about 8 to about 14 carbon atoms, with (b) glucose or a glucose-containing polymer. The alkyl gluocosides have about 1 to about 6 glucose residues per molecule of alkyl glucoside. A preferred glucoside is decyl glucoside, which is the condensation product of decyl alcohol with a glucose polymer and is available commercially from Henkel Corporation of Hoboken, N.J. under the tradename, “Plantaren 2000.”

Examples of amphoteric surfactants suitable for use in the present invention include, but are not limited to, amphocarboxylates such as alkylamphoacetates (mono or di); alkyl betaines; alkylamidoalkyl betaines; alkylamidoalkyl sultaines; alkylamphophosphates; phosphorylated imidazolines such as phosphobetaines and pyrophosphobetaines; carboxyalkyl alkyl polyamines; alkylimino-dipropionates; alkylamphoglycinates (mono or di); alkylamphoproprionates (mono or di),); N-alkyl β-aminoproprionic acids; alkylpolyamino carboxylates; and mixtures thereof. As used herein, the term “amphoteric” shall mean: 1) molecules that contain both acidic and basic sites such as, for example, an amino acid containing both amino (basic) and acid (e.g., carboxylic acid, acidic) functional groups; or 2) zwitterionic molecules which possess both positive and negative charges within the same molecule. The charges of the latter may be either dependent on or independent of the pH of the composition. The amphoteric surfactants are disclosed herein without a counter ion. One skilled in the art would readily recognize that under the pH conditions of the compositions of the present invention, the amphoteric surfactants are either electrically neutral by virtue of having balanced positive and negative charges, or they have counter ions such as alkali metal, alkaline earth, or ammonium counter ions.

Classes of cationic surfactants include alkyl quaternaries (mono, di, or tri), benzyl quaternaries, ester quaternaries, ethoxylated quaternaries, alkyl amines, and mixtures thereof, wherein the alkyl group has from about 6 carbon atoms to about 30 carbon atoms, with about 8 to about 22 carbon atoms being preferred. These cationic surfactants can be employed in composition of the present invention in an amount, based upon the total weight of the composition, from about 0.01% to about 18%, or from about 0.05% to about 15% or from about 0.1% to about 10%.

In one or more embodiments, the emulsifier comprises a liquid crystal emulsifier. In one or more embodiments, the emulsifier is selected from the group consisting of sorbitan olivate, ceterayl olivate, C₁₂-20 alkyl glucoside, C₁₄-22 alcohols, sorbitan stearate, sorbityl laurate, polyglyceryl-6 pentaoleate, and combinations thereof. Suitable emulsifier are available under tradenames Montanov L (INCI-C12-20 Alkyl Glucoside, C14-22 Alcohols), Arlacel LC (INCI-Sorbitan Stearate (and) Sorbityl Laurate), and Oleamuls® WO: (INCI-Polyglyceryl-6 Pentaoleate).

The emulsifier may be present in an amount of from about 0.1% to about 5%, about 0.1 to about 4.5%, about 0.1% to about 4%, about 0.2% to about 3.5%, about 0.2% to about 3%, about 0.2% to about 2%, about 0.5% to about 1.7%, or about 0.5% to about 1.5% by weight % of the total composition. In one or more embodiments, the emulsifier is selected from the group consisting of sorbitan olivate, ceterayl olivate and combinations thereof, and is present in an amount of from about 0.1% to about 5%, about 0.1 to about 4.5%, about 0.1% to about 4%, about 0.2% to about 3.5%, about 0.2% to about 3%, about 0.2% to about 2%, about 0.5% to about 1.7%, or about 0.5% to about 1.5% by weight % of the total composition.

### Humectant

As used herein, “humectant” refers to a compound intended to increase the water content of the top layers of skin (e.g., hygroscopic compounds). Examples of suitable humectants include those found in Chapter 35, pages 399-415 (Skin Feel Agents, by G Zocchi) in Handbook of Cosmetic Science and Technology (edited by A. Barel, M. Paye and H. Maibach, Published in 2001 by Marcel Dekker, Inc New York, NY) and include, but are not limited to, glycerin, sorbitol or trehalose (e.g., α,α-trehalose, β,β-trehalose, α,β-trehalose) or a salt or ester thereof (e.g., trehalose 6-phosphate).

Any of a variety of commercially available humectants, are suitable for use in the present invention. What is meant by a humectant is a compound intended to increase the water content of the top layers of skin (e.g., hygroscopic compounds). Examples of suitable humectants include those found in Chapter 35, pages 399-415 (Skin Feel Agents, by G Zocchi) in Handbook of Cosmetic Science and Technology (edited by A. Barel, M. Paye and H. Maibach, Published in 2001 by Marcel Dekker, Inc New York, NY) and include, but are not limited to, glycerin, sorbitol or trehalose (e.g., α,α-trehalose, β,β-trehalose, α,β-trehalose) or a salt or ester thereof (e.g., trehalose 6-phosphate).

In further embodiments, the humectant comprises a polar humectant. In further embodiments, the humectant is selected from the group consisting of glycerin, sorbitol, xylitol, hyaluronic acid, lactic acid, and combinations thereof.

The humectant may be present in an amount of from about 0.5% to about 15%, about 0.5 to about 14%, about 0.8% to about 13%, about 1% to about 12%, about 2% to about 12%, about 2% to about 10%, about 2.5% to about 8.5%, about 3% to about 8%, about 2.5% to about 3.5%, or 7.5% to about 8.5% by weight % of the total composition. In one or more embodiments, the humectant comprises glycerin, and is present in an amount of from about 0.5% to about 15%, about 0.5 to about 14%, about 0.8% to about 13%, about 1% to about 12%, about 2% to about 12%, about 2% to about 10%, about 2.5% to about 8.5%, about 3% to about 8%, about 2.5% to about 3.5%, or 7.5% to about 8.5% by weight % of the total composition.

### Compound of Formula I

As noted above, the compounds of Formula I is represented by the following structure:

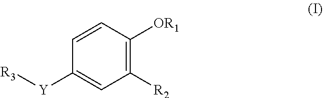

wherein:
   R₁ is selected from the group consisting of C₁-C₂₀ alkyl, C₂-C₂₀ alkenyl, C₂-C₂₀ alkynyl, and C₃-C₈ cycloalkyl or aryl;
   R₂ is selected from the group consisting of hydrogen, hydroxyl, C₁-C₆ alkyl, C₂-C₆ alkenyl, C₂-C₆ alkynyl, C₃-C₈ cycloalkyl or aryl, —OC₁-C₆ alkyl, —OC₂-C₆ alkenyl, —OC₂-C₆ alkynyl, —OC₃-C₈ cycloalkyl or aryl, thiol, —SC₁-C₆ alkyl, —SC₂-C₆ alkenyl, —SC₂-C₆ alkynyl, —SC₃-C₈ cycloalkyl or aryl, —NR₄C₁-C₆ alkyl, —NR₄C₂-C₆ alkenyl, —NR₄C₂-C₆ alkynyl, and —NR₄C₃-C₈ cycloalkyl or aryl;
   R₃ is selected from —CO₂H, —CO₂R₄ or an isosteric equivalent of a carboxy group, wherein R₄ is C₁-C₆ alkyl, C₂-C₆ alkenyl, C₃-C₈ cycloalkyl or aryl; and
Y is —(CH₂—CH₂)—, —(CH═CH)—, or —(C═C)—;
   or a cosmetically acceptable salt thereof.

In one or more embodiments, R₁ is selected from the group consisting of C₅-C₁₆ alkyl, C₅-C₁₆ alkenyl, and C₅-C₁₆ alkynyl; R₂ is selected from the group consisting of hydrogen, hydroxyl, —OC₁-C₆ alkyl, —OC₂-C₆ alkenyl, —OC₂-C₆ alkynyl, —OC₃-C₈ cycloalkyl; R₃ is selected from —CO₂H, —CO₂R₄ wherein R₄ is C₁-C₆ alkyl, or an isosteric equivalent of a carboxy group; and Y is —(CH₂—CH₂)— or —(CH═CH)— (or a cosmetically acceptable salt thereof). In some embodiments, R₁ is selected from the group consisting of C₅-C₁₆ alkenyl; and R₂ is selected from the group consisting of hydrogen or —OC₁-C₃ alkyl. In one or more embodiments, the compound of Formula I is selected from the group consisting of 3-(4-farnesyloxyphenyl)-propionic acid, 3-(4-farnesyloxy-3-hydroxyphenyl)-propionic acid, 3-(4-farnesyloxy-3-methoxyphenyl)-propionic acid, ethyl esters thereof, and combinations of two or more thereof. In one or more embodiments, the compound of the above Formula I is 3-(4-farnesyloxyphenyl)-propionic acid and/or its ethyl ester. In preferred embodiments, the compound of Formula I is comprises 3-(4-farnesyloxyphenyl)-propionic acid. The 3-(4-farnesyloxyphenyl)-propionic acid and/or its ethyl ester can be synthesized using conventional organic synthesis processes.

The compound of Formula I may be present in an amount ranging from about 0.00001% to 10%, or about 0.0001 to about 10%, or about 0.001 to about 5%, or about 0.001% to about 1%, or about 0.01% to about 3%, about 0.01% to about 1%, about 0.01% to about 0.5%, or about 0.005% to about 1.5%, or about 0.005% to about 0.06%, or about 0.009% to about 0.06%, or about 0.009% to about 0.03% by total weight of the composition.

Compounds according to Formula I can also be obtained from natural sources. For example, a compound according to Formula I may be found in a botanical extract. Accordingly, the composition may comprise a botanical extract comprising the compound of Formula I.

In one or more embodiments, the botanical extract is an extract of a plant of the genus Acronychia. In further embodiments, the botanical extract is an extract of Acronychia acidula (also known as lemon aspen). In one or more embodiments, at least one compound of the above Formula I is present in the extract of Acronychia at a concentration equal to or greater than about 0.01 to about 30%, or about 0.1% to about 30%, or about 0.1 to about 20%, or about 1% to about 20%, or about 1% to about 10%, or about 1.5% to about 9%, or about 3% to about 9%, by weight of the Acronychia extract. In further embodiments, the extract of Acronychia acidula comprises 3-(4-farnesyloxyphenyl)-propionic acid in a concentration ranging from about 1% to about 10% by total weight of the extract.

Suitable extracts may be obtained using conventional methods including, but not limited to, direct extraction of material from the biomass by grinding, macerating, pressing, squeezing, mashing, centrifuging, and/or processes such as cold percolation, agitation/distillation, microwave assisted extraction, supercritical/subcritical CO₂ compressed gas extraction with or without polar modifiers, pressurized solvent extraction, accelerated solvent extraction, pressurized or normal hot water extraction, surfactant assisted pressurized hot water extraction, oil extraction, membrane extraction, Soxhlet extraction, the gold finger distillation/extraction and/or processes disclosed, for example, in U.S. Pat. Nos. 7,442,391, 7,473,435, and 7,537,791 to Integrated Botanical Technologies, LLC, incorporated herein by reference, and the like, or by other methods such as solvent extraction, and the like.

Any of a variety of solvents including polar solvents, non-polar solvents, or combinations of two or more thereof may be used in methods comprising solvent extraction. Suitable polar solvents include polar inorganic solvents such as water and the like, polar organic solvents such as alcohols and corresponding organic acids, for example C₁-C₈ alcohols including methanol, ethanol, propanol, butanol, and the like and organic acids, including acetic acid, formic acid, propanoic acid, and the like, polyols and glycols, including C₁-C₈ polyols/glycols and the like, and combinations of two or more thereof. Suitable non-polar solvents include non-polar organic solvents such as alkanes, including C₁-C₈ alkanes, cycloalkanes, including C₁-C₈ alkanes, alkyl ethers, including C₁-C₈ alkyl ethers, Petroleum ethers, ketones, including C₁-C₈ ketones, methylene chloride, ethyl acetate, xylene, toluene, chloroform, vegetable oil, mineral oil and the like. In another embodiment extraction may be obtained by non-polar solvents described above or supercritical fluid extraction with or without a polar modifier such as C₁-C₈ alcohols, water, C₁-C₈ polyols/glycols or C₁-C₈ organic acids.

In one or more embodiments, the extract comprises an extract of Acronychia acidula. In some embodiments, the extract of the invention comprises a combination of polar and non-polar extracts of Acronychia acidula fruit. In another embodiment, the extract of the invention comprises alcoholic or glycolic extracts of Acronychia acidula fruit.

In one or more embodiments, the extract is a polar extract. In further embodiments, the extract is a polar extract prepared using a polar solvent comprising water, C₁-C₈ alcohols, C₁-C₈ polyols, or C₁-C₈ glycols, or combinations of two or more thereof. In certain embodiments, the extract is extracted using one or more C₁-C₄ alcohols, C₁-C₄ polyols, and/or C₁-C₄ glycols. In one or more embodiments, the extract is prepared using a solvent comprising methanol, ethanol, or a combination thereof with or without presence of water. In further embodiments, the extract is a polar extract extracted from Acronychia acidula fruit using a combination of alcohol and water.

In one or more embodiments, the extract is a non-polar extract prepared using a non-polar solvent comprising one or more C₁-C₈ alkanes, C₁-C₈ cycloalkanes, C₁-C₈ alkyl ethers, C₁-C₈ alkyl esters and/or chloroform, more preferably one or more C₁-C₈ alkanes, C₁-C₈ alkyl esters and/or chloroform. In further embodiments, extract is a non-polar extract prepared using hexanes, ethyl acetate, chloroform, or mixtures of two or more thereof. In yet further embodiments, the extract is a non-polar extract prepared using ethyl acetate.

For example, an extract using the fruit of Acronychia acidula may be prepared by homogenizing the fruit in a blender for 30 seconds with denatured alcohol in equal part to fruit. The pulp can then mixed and stirred for another 24 hours at ambient temperature (22 to 26 degrees C.).

Additional denatured alcohol may be added as needed to keep the pulp covered well in alcohol. The mixture then can then be gravity filtered, and the resulting filter cake washed with additional amounts of denatured alcohol. The total filtrate then may then be dried under reduced pressure to remove alcohol. The residue can then be freeze dried to obtain dry matter free of extraction solvent and water. The extraction may be repeated on the filter cake a few times with an extract yield of 5-7% regularly obtained from each extraction.

Another example of the preparation of Acronychia acidula fruit extract is as follows: 500 μm of freeze-dried fruits of Acronychia acidula may be sliced into approximately 5 mm cubes and soaked with 5 L of ethanol at a ratio of 1:10 (raw material to solvent) and stirred at room temperature for 12 hours. The suspension may then be filtered and resulting filtrate concentrated under low pressure to afford a concentrate. The concentrate can then further be dried by freeze-drying methods to obtain 325 μm of residual material called crude extract (65% yield). A sample of the crude extract, 200 μm, can then be taken up in 1 L ethanol and stirred at room temperature overnight. The mixture may then filtered and dried at reduced pressure and at low temperature to provide the extract.

The extract may be present in an amount of about 0.00001, 0.0001, 0.001, 0.01, 0.1, 0.2, 0.3, 0.4, 0.5, 0.75, 1, 1.5 or 2 to about 0.00005, 0.0005, 0.005, 0.05, 0.5, 1, 1.5, 2, 2.5, 3, 4 or 5 wt. % by total weight of the composition. In one or more embodiments, the extract is present in an amount of about 0.01 to about 5 wt. % by total weight of the composition. In further embodiments, the extract is present in an amount of about 0.01 to about 3 wt. % by total weight of the composition. In one or more embodiments, the extract is present in an amount of about 0.1 to about 3 wt. % by total weight of the composition. In further embodiments, the extract is present in an amount of about 0.2 to about 2.5 wt. % by total weight of the composition. In one or more embodiments, the extract is present in an amount of about 0.5 to about 2 wt. % by total weight of the composition. In further embodiments, the extract is present in an amount of about 0.5 to about 1.5 wt. % by total weight of the composition.

The above embodiments may be combined in any suitable combination. For example, in an exemplary embodiment, the skincare composition comprises
a. dimethicone;
b. a viscosity increaser selected from the group consisting of polyacrylate crosspolymer-6, polyacrylate-13, sorbitan stearate, and combinations thereof;
c. an emulsifier selected from the group consisting of sorbitan olivate, ceterayl olivate, and combinations thereof;
d. glycerin; and
e. a polar botanical extract of Acronychia acidula.

### Other Ingredients

Any suitable carrier may be used in the compositions of the present invention. Preferably, for a skin care composition, the carrier is a cosmetically-acceptable carrier. As will be recognized by those of skill in the art, cosmetically-acceptable carriers comprise carriers that are suitable for use in contact with the body, in particular the skin for antiaging applications, without undue toxicity, incompatibility, instability, irritation, allergic response, and the like. A safe and effective amount of carrier is from about 50% to about 99.999%, preferably from about 80% to about 99.9%, more preferably from about 99.9% to about 95%, most preferably from about 99.8% to about 98% of the composition. The carrier can be in a wide variety of forms. For example, emulsion carriers, including, but not limited to, oil-in-water, water-in-oil, water-in-oil-in-water, and oil-in-water-in-silicone emulsions, are useful herein. These emulsions can cover a broad range of viscosities, e.g., from about 100 cP to about 200,000 cP. Examples of suitable cosmetically-acceptable carriers include cosmetically-acceptable solvents and materials for cosmetic solutions, suspensions, lotions, creams, serums, essences, gels, toners, sticks, sprays, ointments, liquid washes and soap bars, shampoos, hair conditioners, pastes, foams, mousses, powders, shaving creams, wipes, patches, strips, powered patches, microneedle patches, bandages, hydrogels, film-forming products, facial and skin masks, makeup, liquid drops, and the like. These product types may contain several types of cosmetically-acceptable carriers including, but not limited to solutions, suspensions, emulsions such as microemulsions and nanoemulsions, gels, solids, liposomes, other encapsulation technologies and the like. In one or more embodiments, the composition is in the form of a solution, suspension, emulsion, lotion, cream, serum, gel, stick, spray, ointment, liquid wash, soap bar, shampoo, hair conditioner, paste, foam, powder, mousse, shaving cream, hydrogel, or film-forming product.

The following are non-limitative examples of such carriers. Other carriers can be formulated by those of ordinary skill in the art. In one embodiment, the carrier contains water. In a further embodiment, the carrier may also contain one or more aqueous or organic solvents. Examples of organic solvents include, but are not limited to: dimethyl isosorbide; isopropylmyristate; surfactants of cationic, anionic and nonionic nature; vegetable oils; mineral oils; waxes; gums; synthetic and natural gelling agents; alkanols; glycols; and polyols. Examples of glycols include, but are not limited to, glycerin, propylene glycol, butylene glycol, pentylene glycol, hexylene glycol, polyethylene glycol, polypropylene glycol, diethylene glycol, triethylene glycol, capryl glycol, glycerol, butanediol and hexanetriol, and copolymers or mixtures thereof. Examples of alkanols include, but are not limited to, those having from about 2 carbon atoms to about 12 carbon atoms (e.g., from about 2 carbon atoms to about 4 carbon atoms), such as isopropanol and ethanol. Examples of polyols include, but are not limited to, those having from about 2 carbon atoms to about 15 carbon atoms (e.g., from about 2 carbon atoms to about 10 carbon atoms) such as propylene glycol. The organic solvents may be present in the carrier in an amount, based upon the total weight of the carrier, of from about 1 percent to about 99.99 percent (e.g., from about 20 percent to about 50 percent). Water may be present in the carrier (prior to use) in an amount, based upon the total weight of the carrier, of from about 5 percent to about 95 percent (e.g., from about 50 percent to about 90 percent). Solutions may contain any suitable amounts of solvent, including from about 40 to about 99.99%. Certain preferred solutions contain from about 50 to about 99.9%, from about 60 to about 99%, from about 70 to about 99%, from about 80 to about 99%, or from about 90 to 99%.

A lotion can be made from such a solution. Lotions typically contain at least one emollient in addition to a solvent. Lotions may comprise from about 1% to about 20% (e.g., from about 5% to about 10%) of an emollient(s) and from about 50% to about 90% (e.g., from about 60% to about 80%) of water. As used herein, “emollients” refer to materials used for the prevention or relief of dryness, as well as for the protection of the skin or hair. Examples of emollients include, but are not limited to, those set forth in the International Cosmetic Ingredient Dictionary and Handbook, eds. Wenninger and McEwen, pp. 1656-61, 1626, and 1654-55 (The Cosmetic, Toiletry, and Fragrance Assoc., Washington, D.C., 7th Edition, 1997) (hereinafter “ICI Handbook”).

Another type of product that may be formulated from a solution is a cream. A cream typically contains from about 5% to about 50% (e.g., from about 10% to about 20%) of an emollient(s) and from about 45% to about 85% (e.g., from about 50% to about 75%) of water.

Yet another type of product that may be formulated from a solution is an ointment. An ointment may contain a simple base of animal, vegetable, or synthetic oils or semi-solid hydrocarbons. An ointment may contain from about 2% to about 10% of an emollient(s) plus from about 0.1% to about 2% of a thickening agent(s).

The compositions useful in the present invention can also be formulated as emulsions. If the carrier is an emulsion, from about 1% to about 10% (e.g., from about 2% to about 5%) of the carrier contains an emulsifier(s). Emulsifiers may be nonionic, anionic or cationic. Examples of emulsifiers include, but are not limited to, those set forth in the ICI Handbook, pp. 1673-1686.

Lotions and creams can be formulated as emulsions. Typically such lotions contain from 0.5% to about 5% of an emulsifier(s), while such creams would typically contain from about 1% to about 20% (e.g., from about 5% to about 10%) of an emollient(s); from about 20% to about 80% (e.g., from 30% to about 70%) of water; and from about 1% to about 10% (e.g., from about 2% to about 5%) of an emulsifier(s).

Single emulsion skin care preparations, such as lotions and creams, of the oil-in-water type and water-in-oil type are well-known in the art and are useful in the subject invention. Multiphase emulsion compositions, such as the water-in-oil-in-water type or the oil-in-water-in-oil type, are also useful in the subject invention. In general, such single or multiphase emulsions contain water, emollients, and emulsifiers as essential ingredients.

The compositions of this invention can also be formulated as a gel (e.g., an aqueous, alcohol, alcohol/water, or oil gel using a suitable gelling agent(s)). Suitable gelling agents for aqueous and/or alcoholic gels include, but are not limited to, natural gums, acrylic acid and acrylate polymers and copolymers, and cellulose derivatives (e.g., hydroxymethyl cellulose and hydroxypropyl cellulose). Suitable gelling agents for oils (such as mineral oil) include, but are not limited to, hydrogenated butylene/ethylene/styrene copolymer and hydrogenated ethylene/propylene/styrene copolymer. Such gels typically contains between about 0.1% and 5%, by weight, of such gelling agents.

The compositions of the present invention can also be formulated into a solid formulation (e.g., a wax-based stick, soap bar composition, powder, or wipe). The composition of the present invention can also be combined with a solid, semi-solid or dissolvable substrate (e.g., a wipe, mask, pad, glove or strip).

The compositions of the present invention may further comprise any of a variety of additional cosmetically active agents, although they are preferably formulated to account for use on skin. Examples of suitable additional active agents include: additional skin lightening agents, darkening agents, anti-acne agents, shine control agents, antimicrobial agents such as anti-yeast agents, anti-fungal, and anti-bacterial agents, anti-inflammatory agents, anti-parasite agents, external analgesics, sunscreens, photo-protectors, antioxidants, keratolytic agents, detergents/surfactants, moisturizers, nutrients, vitamins, energy enhancers, anti-perspiration agents, astringents, deodorants, hair removers, hair growth enhancing agents, hair growth delaying agents, firming agents, hydration boosters, efficacy boosters, anti-callous agents, agents for skin conditioning, anti-cellulite agents, fluorides, teeth whitening agents, anti-plaque agents, and plaque-dissolving agents, odor-control agents such as odor masking or pH-changing agents, and the like. Examples of various suitable additional cosmetically acceptable actives include hydroxy acids, benzoyl peroxide, D-panthenol, UV filters such as but not limited to avobenzone (Parsol 1789), bisdisulizole disodium (Neo Heliopan AP), diethylamino hydroxybenzoyl hexyl benzoate (Uvinul A Plus), ecamsule (Mexoryl SX), methyl anthranilate, 4-aminobenzoic acid (PABA), cinoxate, ethylhexyl triazone (Uvinul T 150), homosalate, 4-methylbenzylidene camphor (Parsol 5000), octyl methoxycinnamate (Octinoxate), octyl salicylate (Octisalate), padimate O (Escalol 507), phenylbenzimidazole sulfonic acid (Ensulizole), polysilicone-15 (Parsol SLX), trolamine salicylate, Bemotrizinol (Tinosorb S), benzophenones 1-12, dioxybenzone, drometrizole trisiloxane (Mexoryl XL), iscotrizinol (Uvasorb HEB), octocrylene, oxybenzone (Eusolex 4360), sulisobenzone, bisoctrizole (Tinosorb M), titanium dioxide, zinc oxide, carotenoids, free radical scavengers, spin traps, retinoids and retinoid precursors such as retinol, retinoic acid and retinyl palmitate, ceramides, polyunsaturated fatty acids, essential fatty acids, enzymes, enzyme inhibitors, minerals, hormones such as estrogens, steroids such as hydrocortisone, 2-dimethylaminoethanol, copper salts such as copper chloride, peptides containing copper such as Cu:Gly-His-Lys, coenzyme Q10, amino acids such a proline, vitamins, lactobionic acid, acetyl-coenzyme A, niacin, riboflavin, thiamin, ribose, electron transporters such as NADH and FADH₂, and other botanical extracts such as oat, aloe vera, Feverfew, Soy, Shiitake mushroom extracts, and derivatives and mixtures thereof.

In one or more embodiments, the compositions of the present invention are skin care compositions that comprise a compound of Formula I and at least one skin lightening active agent. Examples of suitable skin lightening active agents include, but are not limited to, tyrosinase inhibitors, melanin-inhibiting agents, melanosome transfer inhibiting agents including PAR-2 antagonists, exfoliants, sunscreens, retinoids, antioxidants, Tranexamic acid, skin bleaching agents, allantoin, opacifiers, talcs and silicas, zinc salts, and the like, and other agents as described in Solano et al. Pigment Cell Res. 2006, 19 (550-571). Examples of suitable tyrosinase inhibitors include but, are not limited to, Vitamin C and its derivatives, Vitamin E and its derivatives, Kojic Acid, Arbutin, resorcinols, hydroquinone, Flavones e.g. Licorice flavanoids, Licorice root extract, Mulberry root extract, Dioscorea Coposita root extract, Saxifraga extract and the like, Ellagic acid, Salicylates and derivatives, Glucosamine and derivatives, Fullerene, Hinokitiol, Dioic acid, Acetyl glucosamine, Magnolignane, combinations of two or more thereof, and the like. Examples of vitamin C derivatives include, but are not limited to, ascorbic acid and salts, Ascorbic Acid-2-Glucoside, sodium ascorbyl phosphate, magnesium ascorbyl phosphate, and natural extract enriched in vitamin C. Examples of vitamin E derivatives include, but are not limited to, alpha-tocopherol, beta, tocopherol, gamma-tocopherol, delta-tocopherol, alpha-tocotrienol, beta-tocotrienol, gamma-tocotrienol, delta-tocotrienol and mixtures thereof, tocopherol acetate, tocopherol phosphate and natural extracts enriched in vitamin E derivatives. Examples of resorcinol derivatives include, but are not limited to, resorcinol, 4-substituted resorcinols like 4-alkylresorcinols such as 4-butyresorcinol (rucinol), 4-hexylresorcinol, phenylethyl resorcinol, 1-(2,4-dihydroxyphenyl)-3-(2,4-dimethoxy-3-methylphenyl)-Propane and the like and natural extracts enriched in resorcinols. Examples of salicylates include, but are not limited to, salicylic acid, acetylsalicylic acid, 4-methoxysalicylic acid and their salts. In certain preferred embodiments, the tyrosinase inhibitors include a 4-substituted resorcinol, a vitamin C derivative, or a vitamin E derivative. In more preferred embodiments, the tyrosinase inhibitor comprises Phenylethyl resorcinol, 4-hexyl resorcinol, or ascorbyl-2-glucoside.

Examples of suitable melanin-degradation agents include, but are not limited to, peroxides and enzymes such as peroxidases and ligninases. In certain preferred embodiments, the melanin-inhibiting agents include a peroxide or a ligninase.

Examples of suitable melanosome transfer inhibiting agents including PAR-2 antagonists such as soy trypsin inhibitor or Bowman-Birk Inhibitor, Vitamin B3 and derivatives such as Niacinamide, Essential soy, Whole Soy, Soy extract. In certain preferred embodiments, the melanosome transfer inhibiting agents includes a soy extract or niacinamide.

Examples of exfoliants include, but are not limited to, alpha-hydroxy acids such as lactic acid, glycolic acid, malic acid, tartaric acid, citric acid, or any combination of any of the foregoing, beta-hydroxy acids such as salicylic acid, polyhydroxy acids such as lactobionic acid and gluconic acid, and mechanical exfoliation such as microdermabrasion. In certain preferred embodiments, the exfoliant include glycolic acid or salicylic acid.

Examples of sunscreens include, but are not limited to, avobenzone (Parsol 1789), bisdisulizole disodium (Neo Heliopan AP), diethylamino hydroxybenzoyl hexyl benzoate (Uvinul A Plus), ecamsule (Mexoryl SX), methyl anthranilate, 4-aminobenzoic acid (PABA), cinoxate, ethylhexyl triazone (Uvinul T 150), homosalate, 4-methylbenzylidene camphor (Parsol 5000), octyl methoxycinnamate (Octinoxate), octyl salicylate (Octisalate), padimate O (Escalol 507), phenylbenzimidazole sulfonic acid (Ensulizole), polysilicone-15 (Parsol SLX), trolamine salicylate, Bemotrizinol (Tinosorb S), benzophenones 1-12, dioxybenzone, drometrizole trisiloxane (Mexoryl XL), iscotrizinol (Uvasorb HEB), octocrylene, oxybenzone (Eusolex 4360), sulisobenzone, bisoctrizole (Tinosorb M), titanium dioxide, zinc oxide, and the like.

Examples of retinoids include, but are not limited to, retinol, retinaldehyde, retinoic acid, retinyl palmitate, isotretinoin, tazarotene, bexarotene and Adapalene. In certain preferred embodiments, the retinoid is retinol. Nevertheless, in one or more embodiments, the composition is essentially free of retinoids or retinoid precursors; and in further embodiments, is free of retinoids or retinoid precursors.

Examples of antioxidants include, but are not limited to, water-soluble antioxidants such as sulfhydryl compounds and their derivatives (e.g., sodium metabisulfite and N-acetyl-cysteine, glutathione), lipoic acid and dihydrolipoic acid, stilbenoids such as resveratrol and derivatives, lactoferrin, and ascorbic acid and ascorbic acid derivatives (e.g., ascobyl-2-glucoside, ascorbyl palmitate and ascorbyl polypeptide). Oil-soluble antioxidants suitable for use in the compositions of this invention include, but are not limited to, butylated hydroxytoluene, retinoids (e.g., retinol and retinyl palmitate), tocopherols (e.g., tocopherol acetate), tocotrienols, and ubiquinone. Natural extracts containing antioxidants suitable for use in the compositions of this invention, include, but not limited to, extracts containing flavonoids and isoflavonoids and their derivatives (e.g., genistein and diadzein), extracts containing resveratrol and the like. Examples of such natural extracts include grape seed, green tea, pine bark, feverfew, parthenolide-free feverfew, oat extracts, pomelo extract, wheat germ extract, Hesperidin, Grape extract, Portulaca extract, Licochalcone, chalcone, 2,2′-dihydroxy chalcone, Primula extract, propolis, and the like.

The additional cosmetically active agent may be present in a composition in any suitable amount, for example, in an amount of from about 0.0001% to about 20% by weight of the composition, e.g., about 0.001% to about 10% such as about 0.01% to about 5%. In certain preferred embodiments, in an amount of 0.1% to 5% and in other preferred embodiments from 1% to 2%.

A variety of other materials may also be present in the compositions of the present invention. These include, for example, chelating agents, humectants, opacifiers, conditioners, preservatives, fragrances and the like. The compositions may include surfactants, for example, those selected from the group consisting of anionic, non-ionics, amphoteric, cationic, or a combination of two or more thereof.

The compositions of the present invention may also contain chelating agents (e.g., EDTA) and preservatives (e.g., parabens). Examples of suitable preservatives and chelating agents are listed in pp. 1626 and 1654-55 of the ICI Handbook. In addition, the compositions useful herein can contain conventional cosmetic adjuvants, such as colorants such as dyes and pigments, opacifiers (e.g., titanium dioxide), and fragrances.

In one or more embodiments, the present invention comprises applying a compound or composition of the invention via a substrate comprising such material. Any suitable substrate may be used in the present invention. Examples of suitable substrates and substrate materials are disclosed, for example, in U.S. Published Application Nos. 2005/022683 and 2009/0241242 which are incorporated herein by reference in their entirety. In certain preferred embodiments, the substrate is a wipe or a facial mask.

The composition and products containing such compositions of this invention may be prepared using methodology that is well known by an artisan of ordinary skill.

### Methods

Another aspect of the invention pertains to a method for treating skin, the method comprising topically applying to skin a composition in accordance with one more embodiments of the invention. As used herein, “treatment” or “treating” means the amelioration, prophylaxis, or reversal of a condition, disease, or disorder, or at least one discernible symptom thereof. In one embodiment, “treatment” or “treating” refers to an amelioration, prophylaxis, or reversal of at least one measurable physical parameter related to the condition, disease, or disorder being treated, not necessarily discernible in or by the subject being treated. In another embodiment, “treatment” or “treating” refers to inhibiting or slowing the progression of a condition, disease, or disorder, either physically, e.g., stabilization of a discernible symptom, physiologically, e.g., stabilization of a physical parameter, or both. In another embodiment, “treatment” or “treating” refers to delaying the onset of a condition, disease, or disorder.

The compositions/compounds described herein may be used in treating skin for treating acne, treating the signs of aging (e.g., wrinkles), improving skin barrier function and/or lightening skin. In one or more embodiments, said treatment is for a subject who has a condition or a history of a condition selected from the group consisting of atopic dermatitis, rosacea, seborrheic dermatitis, psoriasis, dry skin, flaky skin.

Compositions of the invention are suitable for improving the texture of skin or improving the firmness of skin, or any of the conditions/symptoms described below.

As used herein, “improving the texture of skin” means the smoothing of the surface of the skin to remove either bumps or crevasses on the skin surface.

As used herein, “improving the firmness of skin” means the enhancing of the firmness or elasticity of the skin, preventing the loss of firmness or elasticity of skin, or preventing or treating sagging, lax and loose skin.

As used herein, “loss of elasticity” includes loss of elasticity or structural integrity of the skin or tissue, including but not limited to sagging, lax and loose tissue. The loss of elasticity or tissue structure integrity may be a result of a number of factors, including but not limited to disease, aging, hormonal changes, mechanical trauma, environmental damage, or the result of an application of products, such as a cosmetics or pharmaceuticals, to the tissue.

As used herein, “uneven skin” means a condition of the skin associated with diffuse or mottled pigmentation, which may be classified as hyperpigmentation, such as post-inflammatory hyperpigmentation.

As used herein, “blotchiness” means a condition of the skin associated with redness or erythema.

As used herein, “age spots” means a condition of the skin associated with discrete pigmentation, e.g., small areas of darker pigmentation that may develop on the face as well as the hands.

Signs of skin aging also include the presence of diminished skin thickness, and abnormal or diminished synthesis of collagen, glycosaminoglycans, proteoglycans, elastin, or glycoproteins including fibronectin. In one embodiment, the sign of aging is selected from the abnormal or diminished synthesis of collagen, glycosaminoglycans, proteoglycans, elastin, or glycoproteins including fibronectin. In another embodiment, the sign of skin aging is diminished synthesis of collagen or elastin.

Examples of skin aging that may be treated by topical use of the compositions of this invention include, but are not limited to, wrinkles on the skin. As used herein, the term “wrinkle” includes fine line, fine wrinkles, coarse wrinkles, cellulite, scars, and stretch marks. Examples of wrinkles include, but are not limited to, fine lines around the eyes (e.g., “crow's feet”), forehead and cheek wrinkles, frown-lines, and laugh-lines around the mouth.

As used herein, “topical use” and “topically applying” means directly laying on or spreading on the skin, hair, or nail, e.g., by use of the hands or an applicator such as a wipe.

The compositions are also suitable for treating or preventing acne. As used herein, “acne” refers to disorders resulting from the actions of hormones and other substances on the sebaceous glands and hair follicles, typically leading to clogged pores and the formation of inflammatory or non-inflammatory lesions on the skin. Specifically, it relates to blemishes, lesions, or pimples, pre-emergent pimples, blackheads, and/or whiteheads. As used herein, a “pre-emergent pimple” is an inflamed follicle that is not visually apparent on the surface of the skin with the naked eye (e.g., as a lesion).

The compositions of the invention are also suitable for treating or preventing rosacea. As used herein, “rosacea” means skin with persistent erythema with or without papules, pustules, or nodules.

The compositions of the invention are also suitable for reducing epidermal hyperkeratinzation. Accordingly, the composition may be used for treatment or prevention of conditions characterized by hyperkeratinzation, such as acne or warts.

In one or more embodiments, one or more of the methods described herein modify the expression of one or more biomarkers. For example, in one or more embodiments, the method may be a method of increasing CRABP2, HAS3, or HBEGF expression in skin. CRABP2 is well-established in literature as a highly sensitive marker of retinoid bioactivity and potency (Elder J T, Cromie M A, Griffiths C E M, Chambon P, Voorhees J J. Stimulus-selective induction of CRABP-II mRNA: A marker for retinoic acid action in human skin. J Invest Dermatol. 1993; 100 (4), and has been associated with conferring skin anti-aging benefits (Bielli A, Scioli M G, D'Amico F, Tarquini C, Agostinelli S, Costanza G, Doldo E, Campione E, Passeri D, Coniglione F, Orlandi A. Cellular retinoic acid binding protein-II expression and its potential role in skin aging. Aging (Albany NY). 2019 Mar. 18; 11 (6): 1619-1632). HAS3 is the hyaluronic acid synthase gene that produces hyaluronic acid, which is associated with skin hydration and plumping and is indirectly induced by retinol. Skin plumpness confers antiaging or youthful appearance. Heparin-binding epidermal growth factor (HbEGF). HbEGF is a marker of cell proliferation, which is a feature associated with retinol.

The compositions described herein may be applied to any skin in need of treatment on the human body. For example, application may be made to any one or more of the skin of the face, neck, chest, back, arms, axilla, hands and/or legs. In certain preferred embodiments, the method comprises applying a composition according to one or more embodiments of the invention to skin of the face.

Any suitable method of applying the extract to the skin in need may be used in accordance with the present invention. For example, the extract may be applied directly from a package to the skin in need, by hand to the skin in need, or may be transferred from a substrate such as a wipe or mask, or a combination of two or more thereof. In other embodiments, the extract may be applied via a dropper, tube, roller, spray, patch or added to a bath or otherwise to water to be applied to the skin, and the like.

In one or more embodiments, the methods of the present invention further comprise the step of leaving the composition in contact with the skin for period of time. For example, in certain preferred embodiments after application, the compound is left in contact with the skin for a period of about 15 minutes or greater. In certain more preferred embodiments, the extract is left in contact with the skin for about 20 minutes or greater, more preferably about 1 hour or greater.

In some embodiments, the method of the present invention comprises a regimen comprising applying the composition to skin multiple times over a selected period of time. For example, in certain embodiments, the present invention provides a method of treating signs of aging comprising applying to skin in need of antiaging a composition in accordance with one or more embodiments of the invention once or twice daily for at least 12 weeks, preferably at least 8 weeks and more preferably for at least 2 weeks.

### EXAMPLES

The below examples evaluate compositions including Acronychia Acidula Fruit extract. The examples pertain to evaluation of the of retinol-responsive genes such as CRABP2, HBEGF, and HAS3. The upregulation of these genes is associated with skin benefits provided by retinoids (e.g., anti-aging effects, reduction of wrinkles, acne, and tone benefits.

### Example 1: Composition Preparation

Five inventive compositions (Ex. 1-5) and three comparative compositions (Ex. 6-8) were prepared in the following manner using the amounts shown in Table 1 and 2 below. Ex. 1-5 were prepared with Acronychia Acidula Fruit extract. Comparative Ex. 7-8 were compositions prepared without Acronychia Acidula Fruit extract. Compositions were prepared by adding purified water to a suitable sized beaker. Mixing was started using a propeller mixing blade and the water was heated to 75-80° C. Sepimax Zen™ was slowly added into the main batch and mixed until fully hydrated and free of visible particles, with the mixing speed increased as needed. The mixture was reheated to 75-80° C., then chlorphenesin and emulsifier were added and mixed until uniform and free of undissolved particles. Next, the solution was allowed to cool to 60-65° C., with the addition of glycerin, and mixed until uniform. Once the solution cooled to 55-60° C., Sepiplus™ 400 and Acronychia Acidula fruit extract (except for Comparative Ex. 7-8) were added and mixed until uniform. Cooling continued to 40° C. while the solution was mixed at moderate speed. Then dimethicone, phenyoxyethanol, and ethylhexylglycerin were added and mixed until uniform. Water was added q.s. and the pH was adjusted. The solution was mixed until uniform and allowed to cool to room temperature. Comparative Ex. 6 is Acronychia Acidula Fruit Extract (30% extract in carrier) purchased from Southern Cross Botanicals Pty Ltd., Knockrow NSW, Australia. Acronychia Acidula fruit extract used in the examples typically contains between about 1% and 10% 3-(4-farnesyloxyphenyl)-propionic acid by weight of the extract with carrier, more particularly between about 1% and about 6%, 0.01 to 0.06% by total weight of formula.

TABLE 1
Inventive Examples

| | Ex. 1 | Ex. 2 | Ex. 3 | Ex. 4 | Ex. 5 |
|---|---|---|---|---|---|
| | 0.5% | 1.0% | 2% | 1% | 1.0% |
| | Lemon | Lemon | Lemon | Lemon | Lemon |
| | Aspen | Aspen | Aspen | Aspen | Aspen |
| | Extract; | Extract; | Extract; | Extract; | Extract; |
| INCI | pH 5 | pH 5 | pH 5 | pH 3 | pH 5 |
| Acronychia Acidula Fruit Extract | 1.67 | 3.34 | 6.67 | 3.34 | 3.34 |
| (30% extract in carrier) | | | | | |
| Sorbitan Olivate (Emulsifier) | 0.20 | 0.20 | 0.20 | 0.20 | 0.20 |
| Cetearyl Olivate (Emulsifier) | 0.30 | 0.30 | 0.30 | 0.30 | 0.30 |
| Sepimax Zen ™ INCI: | 0.80 | 0.80 | 0.80 | 0.80 | 0.80 |
| Polyacrylate Crosspolymer-6 | | | | | |
| (viscosity increaser) | | | | | |
| Sepiplus ™ 400 INCI: | 1.49 | 1.49 | 1.49 | 1.49 | 1.49 |
| Polyacrylate-13 and | | | | | |
| Polyisobutene and Polysorbate 20 | | | | | |
| (viscosity increaser) | | | | | |
| Preservatives | 1 | 1 | 1 | 1 | 1 |
| Glycerin (Humectant) | 3.00 | 3.00 | 3.00 | 3.00 | 8.00 |
| Dimethicone (Xiameter PMX | 1.5 | 1.5 | 1.5 | 1.5 | 3.5 |
| 200) (Emollient) | | | | | |
| Dimethicone and Dimethicone | | | | | 1 |
| cross polymer (Dow Corning | | | | | |
| 9041elastomer blend) (Emollient) | | | | | |
| Water | q.s to 100 | q.s to 100 | q.s to 100 | q.s to 100 | q.s to 100 |
| Sodium Hydroxide | q.s. to pH 5 | q.s. to pH 5 | q.s. to pH 5 | q.s. to pH 3 | q.s. to pH 5 |

TABLE 2
Comparative Examples

| | | Comparative | Comparative |
|---|---|---|---|
| | Comparative | Ex. 7 | Ex. 8 |
| | Ex. 6 | Placebo w/o | Placebo w/o |
| | Lemon Aspen | Lemon Aspen | Lemon Aspen |
| | Extract Alone | Extract; | Extract; |
| INCI | (30% Extract) | pH 5 | pH 3 |
| Acronychia Acidula Fruit Extract (30% | 100.00 | | |
| extract in carrier) | | | |
| Sorbitan Olivate (Emulsifier) | | 0.20 | 0.20 |
| Cetearyl Olivate (Emulsifier) | | 0.30 | 0.30 |
| Sepimax Zen ™ INCI: Polyacrylate | | 0.80 | 0.80 |
| Crosspolymer-6 (viscosity increaser) | | | |
| Sepiplus ™ 400 INCI: Polyacrylate-13 | | 1.49 | 1.49 |
| and Polyisobutene and Polysorbate 20 | | | |
| (viscosity increaser) | | | |
| Preservatives | | 1 | 1 |
| Glycerin (Humectant) | | 3.00 | 3.00 |
| Dimethicone (Emollient) (Xiameter | | 1.5 | 1.5 |
| PMX 200) | | | |
| Water | | q.s to 100 | q.s to 100 |
| Sodium Hydroxide | | q.s. to pH 5 | q.s. to pH 3 |

### Example 2: Bioactivity Assay of CRABP2, HBEGF, and HAS3 in Human Skin Explants

Abdominal skin samples were obtained from human adults undergoing abdominoplasty surgery. Informed consent was obtained from each patient, and all experimental steps were approved by an institutional review board (IRB). Subcutaneous fat was carefully removed and skin biopsies of 0.93 cm² were prepared under sterile conditions and acclimated in DMEM/F12 (1:1) medium, 2% heat-inactivated fetal bovine serum, 10 μg/mL insulin, 10 ng/mL hydrocortisone, 10 ng/mL EGF, 1×ABAM under a 5% CO₂ humidified atmosphere overnight. Skin explants were treated topically with 4 μL of each formulation for 48 hours.

At the end of the 48 h incubation, the skin biopsies were cut in half and either half of the skin biopsies or the two-halves were each lysed in 600 μL lysis buffer, consisting of 100 parts RLT buffer (RNA purification kit, sold under the tradename RNEASY Mini kit, Qiagen, Valencia, CA), to one part 2-mercaptoethanol inside a reinforced tube with screw cap and o-ring closure, and ceramic beads in the tube for tissue grinding (sold under the tradename PRECELLYS CKMix50-R, Bertin Corp, Rockville, MD). The tubes were shaken 40 seconds at 6300 rpm. RNA was extracted from the solutions using the RNEASY Mini Kit (Qiagen, Valencia, CA) according to manufacturer's instructions and RNA was eluted in 30 μL RNase-free water. Each tissue was transferred into hard tissue homogenizing Ozyme CK28 tubes (Bertin Corp, Rockville, MD) containing ceramic beads, 400 μL of RLT buffer, and 4 μL beta-mercaptoethanol and put in ice. After thawing, 300 μL of water and 10 μL of Proteinase K (ThermoFisher Scientific, Bridgewater, NJ) were added in each tube, mixed and incubated 20 min at 55° C.

Reverse transcription (RT) was performed using the Applied Biosystems High Capacity Reverse Transcription Kit (ThermoFisher Scientific, Bridgewater, NJ). Gene expression assays sold under the tradename TAQMAN for cellular retinoic acid binding protein 2 (CRABP2), heparin-binding epidermal growth factor (HBEGF), hyaluronan synthase 3 (HAS3), polymerase (RNA) II polypeptide A (POLR2A), and master mix sold under the tradename TAQMAN (ThermoFisher Scientific, Bridgewater, NJ). qPCR analysis was performed using the TAQMAN master mix, and run on a real time PCR system sold under the tradename QUANTSTUDIO 7 Flex System (ThermoFisher Scientific, Bridgewater, NJ). The expression of these genes was normalized against the expression of the human 18S housekeeping gene. The fold changes were calculated in comparison to the untreated or vehicle controls for CRABP2, HBEGF and HAS3 gene expression, and two-tailed two-sample Student t-tests (Microsoft Office Excel 2007; Microsoft, Redmond, WA, USA) were performed.

TABLE 3
CRABP2 gene expression
CRABP2 Normalized to 18S

| | Avg. Fold | |
|---|---|---|
| Treatment | Change | SD |
| Untreated | 1.01 | 0.14 |
| Ex. 1 - 0.5% LA; pH 5 | 1.20 | 0.29 |
| Ex. 2 - 1% LA; pH 5 | 1.75 | 0.36 |
| Ex. 3 - 2% LA; pH 5 | 2.28 | 0.52 |
| Ex. 5 - 1% LA; pH 5 | 1.67 | 0.25 |
| Ex. 7 - (Comp.) Placebo w/o | 0.57 | 0.06 |
| Lemon Aspen Extract; pH 5 | | |

TABLE 4
HBEGF gene expression
HBEGF Normalized to 18S

| | Avg. Fold | |
|---|---|---|
| Treatment | Change | SD |
| Untreated | 1.04 | 0.16 |
| Ex. 1 - 0.5% LA; pH 5 | 1.42 | 0.11 |
| Ex. 2 - 1% LA; pH 5 | 1.28 | 0.34 |
| Ex. 3 - 2% LA; pH 5 | 2.01 | 0.45 |
| Ex. 5 - 1% LA; pH 5 | 1.62 | 0.25 |
| Ex. 7 - (Comp.) Placebo w/o | 1.09 | 0.13 |
| Lemon Aspen Extract; pH 5 | | |

TABLE 5
HAS3 gene expression
HAS3 Normalized to 18S

| | Avg. Fold | |
|---|---|---|
| Treatment | Change | SD |
| Untreated | 1.01 | 0.18 |
| Ex. 1 - 0.5% LA; pH 5 | 0.82 | 0.15 |
| Ex. 2 - 1% LA; pH 5 | 1.22 | 0.37 |
| Ex. 3 - 2% LA; pH 5 | 1.62 | 0.43 |
| Ex. 5 - 1% LA; pH 5 | 0.97 | 0.19 |
| Ex. 7 - (Comp.) Placebo w/o | 0.64 | 0.04 |
| Lemon Aspen Extract; pH 5 | | |

As seen in Tables 3-4, inventive Ex. 1, 2, 3 and 5 show enhanced expression of CRABP2 and HBEGF gene expression compared to the placebo and untreated control. CRABP2 is well-established in literature as a highly sensitive marker of retinoid bioactivity and potency (Elder J T, Cromie M A, Griffiths C E M, Chambon P, Voorhees J J. Stimulus-selective induction of CRABP-II mRNA: A marker for retinoic acid action in human skin. J Invest Dermatol. 1993; 100 (4), and has been associated with conferring skin anti-aging benefits (Bielli A, Scioli M G, D'Amico F, Tarquini C, Agostinelli S, Costanza G, Doldo E, Campione E, Passeri D, Coniglione F, Orlandi A. Cellular retinoic acid binding protein-II expression and its potential role in skin aging. Aging (Albany NY). 2019 Mar. 18; 11 (6): 1619-1632). Heparin-binding epidermal growth factor (HbEGF) is associated with cell proliferation, one of the hallmarks of retinoid bioactivity. Accordingly, this data shows the inventive examples exhibit an increase of retinol-like property, and hence antiaging benefits.

As seen in Table 5, the is also an increase in HAS3 with respect to the placebo of comparative Ex. 7, and also for Ex. 2-3 with respect to untreated. HAS3, also known as hyaluronic acid 15 synthase 3, encodes for a protein involved in the synthesis of hyaluronic acid. Hyaluronic acid is associated with skin hydration and plumping and is indirectly induced by retinol. Skin plumpness confers antiaging or youthful appearance. Thus upregulation of this gene as seen in the results is also associated with anti-aging and other retinoid behavior.

### Example 3: Bioactivity Assay of CRABP2 and HBEGF in Human Skin Explants (Epidermis Only)

Abdominal skin samples were obtained from human adults undergoing abdominoplasty surgery. Informed consent was obtained from each patient, and all experimental steps were approved by an institutional review board (IRB). Subcutaneous fat was carefully removed and skin biopsies of 0.93 cm² were transferred to 6 well plates. Skin explants were treated topically with 4 μL of each formulation, prepared under sterile conditions and acclimated in Gold Medium supplemented with 1× Natural Cap Vial with Hydrocortisone 0.50 mL, 1× Natural Cap Vial with Transferrin 0.50 mL, 1× Amber Vial with Epinephrine 0.25 mL, 1× Red Cap Vial with Gentamycin Amphotericin-1000 0.50 mL, 1× Orange Cap Vial with BPE 2.00 mL, 1× Green Cap Vial with hEGF 0.50 mL, 1× Lilac Cap Vial with Insulin 0.50 mL (KGM™ Gold Keratinocyte Growth Medium SingleQuots™ Supplements and Growth Factors, (Lonza)), under a 5% CO₂ humidified atmosphere at 37° C. for 48 hours.

At the end of the 48 hour incubation, in order to destroy tissues and homogenize cells, each explant was put in a tube containing 100 μl of water at 60° C. After 50 sec of contact, the epidermis was detached from the dermis and each was transferred separately into Ozyme CK28 tubes containing beads+400 μL of RLT+4 μL beta-mercaptoethanol and put in ice. The Ozyme CK28 tubes containing epidermis tissue were transferred into the tissue grinding device (sold under the tradename PRECELLYS CKMix50-R, Bertin Corp, Rockville, MD) and shaken at 5000 rpm for 60 seconds. After minispin centrifugation (4000 rpm), the content of each tube containing epidermis was transferred in 2 mL Rnase free eppendorf tubes and stored at −80° C. 300 μL of water were added in the tubes containing dermis. After homogenization, the content was transferred in 2 ml Rnase free eppendorf tubes and stored at −80° C. until further processing.

RNA was extracted from the solutions. After thawing, 300 μL of water+10 μL of proteinase K were added in each tube containing epidermal material, mixed and incubated 20 min at 55° C. Using Qiacube HT, RNA was extracted using the epidermis protocol following manufacturer's instructions, and stored at −80° C. Sample was thawed and a nanodrop spectrophotometer was used to assess RNA concentration in 2 μL of RNA extract.

Reverse transcription (RT) was performed using the Applied Biosystems High Capacity Reverse Transcription Kit (ThermoFisher Scientific, Bridgewater, NJ) and real-time PCR detection system sold under the tradename ICYCLER IQ (Bio-Rad Laboratories, Inc., France). Gene expression assays were run for CRABPB2 and HBEGF. qPCR analysis was performed using master mix, sold under the tradename POWER SYBYR Green master mix (ThermoFisher Scientific). The expression of these genes was normalized against the expression of the human 18S housekeeping gene. The fold changes were calculated in comparison to the untreated or vehicle controls and statistical analysis was done using an ANOVA test.

TABLE 6
CRABP2 gene expression
CRABP2 Normalized to 18S

| | Avg. Fold | |
|---|---|---|
| Treatment | Change | SD |
| Untreated | 1 | 0.16 |
| Ex. 2 - 1% LA; pH 5 | 5.44 | 0.25 |
| Ex. 4 - 1% LA; pH 3 | 4.47 | 0.94 |
| Ex. 5 - 1% LA; pH 5 | 3.48 | 0.32 |
| Ex. 6 - LA Alone (Comparative) | 3.04 | 1.24 |
| Ex. 7 - Placebo w/o LA; pH 5 (Comparative) | 1.73 | 0.58 |
| Ex. 8 Placebo w/o LA pH 3 (Comparative) | 1.39 | 0.31 |

TABLE 7
HBEGF gene expression
HBEGF Normalized to 18S

| | Avg. Fold | |
|---|---|---|
| Treatment | Change | SD |
| Untreated | 1 | 0.14 |
| Ex. 2 - 1% LA; pH 5 | 7.79 | 0.64 |
| Ex. 4 - 1% LA; pH 3 | 5.59 | 0.74 |
| Ex. 5 - 1% LA; pH 5 | 4.69 | 0.38 |
| Ex. 6 - LA Alone (Comparative) | 8.94 | 1.52 |
| Ex. 7 - Placebo w/o LA; pH 5 (Comparative) | 2.12 | 0.87 |
| Ex. 8 Placebo w/o LA pH 3 (Comparative) | 2.92 | 0.07 |

As seen from Tables 6-7, inventive Ex. 2, 4 and 5 show enhanced expression of CRABP2 and HBEGF gene expression compared to the placebo and untreated control.

What is claimed is:

1. A skincare composition comprising:
a. an emollient;
b. a viscosity increaser;
c. an emulsifier;
d. a humectant; and
e. a compound of Formula I: 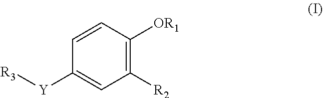
wherein:
R₁ is selected from the group consisting of C₁-C₂₀ alkyl, C₂-C₂₀ alkenyl, C₂-C₂₀ alkynyl, and C₃-C₈ cycloalkyl or aryl;
R₂ is selected from the group consisting of hydrogen, hydroxyl, C₁-C₆ alkyl, C₂-C₆ alkenyl, C₂-C₆ alkynyl, C₃-C₈ cycloalkyl or aryl, —OC₁-C₆ alkyl, —OC₂-C₆ alkenyl, —OC₂-C₆ alkynyl, —OC₃-C₈ cycloalkyl or aryl, thiol, —SC₁-C₆ alkyl, —SC₂-C₆ alkenyl, —SC₂-C₆ alkynyl, —SC₃-C₈ cycloalkyl or aryl, —NR₄C₁-C₆ alkyl, —NR₄C₂-C₆ alkenyl, —NR₄C₂-C₆ alkynyl, and —NR₄C₃-C₈ cycloalkyl or aryl;
R₃ is selected from —CO₂H, —CO₂R₄ or an isosteric equivalent of a carboxy group, wherein R₄ is C₁-C₆ alkyl, C₂-C₆ alkenyl, C₃-C₈ cycloalkyl or aryl; and
Y is —(CH₂—CH₂)—, —(CH═CH)—, or —(C═C)—;
or a cosmetically acceptable salt thereof.

2. The skincare composition of claim 1, wherein R₁ is selected from the group consisting of C₅-C₁₆ alkyl, C₅-C₁₆ alkenyl, and C₅-C₁₆ alkynyl; R₂ is selected from the group consisting of hydrogen, hydroxyl, —OC₁-C₆ alkyl, —OC₂-C₆ alkenyl, —OC₂-C₆ alkynyl, —OC₃-C₈ cycloalkyl; R₃ is selected from —CO₂H, —CO₂R₄ wherein R₄ is C₁-C₆ alkyl, or an isosteric equivalent of a carboxy group; and Y is —(CH₂—CH₂)— or —(CH═CH)—.

3. (canceled)

4. The skincare composition of claim 1, wherein the compound of Formula I is selected from the group consisting of 3-(4-farnesyloxyphenyl)-propionic acid, 3-(4-farnesyloxy-3-hydroxyphenyl)-propionic acid, 3-(4-farnesyloxy-3-methoxyphenyl)-propionic acid, ethyl esters thereof, and combinations of two or more thereof.

5. The skincare composition of claim 4, wherein the compound of Formula I comprises 3-(4-farnesyloxyphenyl)-propionic acid.

6. The skincare composition of claim 1, wherein the concentration of the compound of Formula I is present in an amount ranging from about 0.0001% to about 1% by total weight of the composition.

7. The skincare composition of claim 1, wherein the skincare composition comprises a botanical extract comprising the compound of Formula I, wherein said botanical extract is an extract of Acronychia acidula and is present in an amount ranging from about 0.01% to about 5% by total weight of the composition.

8. (canceled)

9. (canceled)

10. (canceled)

11. (canceled)

12. The skincare composition of claim 11, wherein the extract of Acronychia acidula comprises 3-(4-farnesyloxyphenyl)-propionic acid in a concentration ranging from about 0.01% to about 0.5% by total weight of the composition.

13. The skincare composition of claim 1, wherein the emollient is a silicone or silicone-based emollient.

14. The skincare composition of claim 13, wherein the emollient is selected from the group consisting of dimethicone, dimethicone cross-polymer, phenyl trimethicone, cetyl trimethicone, amodimethicone, caprylyl methicone, and combinations thereof.

15. The skincare composition of claim 13, wherein the emollient is present in a concentration of about 0.1% to about 15% by weight.

16. The skincare composition of claim 1, wherein the viscosity increaser is a rheology modifier that provide yield to the composition and is compatible at pH less than about 5.

17. The skincare composition of claim 16, wherein the viscosity increaser is selected from the group consisting of polyacrylate crosspolymer-6, polyacrylate-13, sorbitan stearate, polyacrylamide, C13-14 isoparaffin, laureth-7, ammonium acryloyldimethyltaurate/VP copolymer, carbomer, and combinations thereof.

18. The skincare composition of claim 1, wherein the viscosity increaser is present in a concentration of about 0.1% to about 5% by weight.

19. The skincare composition of claim 18, wherein the emulsifier comprises a liquid crystal emulsifier.

20. The skincare composition of claim 19, wherein the emulsifier is selected from the group consisting of sorbitan olivate, ceterayl olivate, C12-20 alkyl glucoside, C14-22 alcohols, sorbitan stearate, sorbityl laurate, polyglyceryl-6 pentaoleate, and combinations thereof.

21. The skincare composition of claim 1, wherein the emulsifier is present in a concentration of about 0.1% to about 5% by weight.

22. The skincare composition of claim 1, wherein the humectant comprises a polar humectant.

23. The skincare composition of claim 1, wherein the humectant is selected from the group consisting of glycerin, sorbitol, xylitol, hyaluronic acid, lactic acid, and combinations thereof.

24. The skincare composition of claim 1, wherein the humectant is present in a concentration of about 0.5% to about 15% by weight.

25. A method of treating the skin, the method comprising applying topically to skin the skincare composition of claim 1.

26. The method of claim 25, wherein the method comprises increasing CRABP2 expression in skin cells.

27. A skincare composition comprising:
a. dimethicone;
b. a viscosity increaser selected from the group consisting of polyacrylate crosspolymer-6, polyacrylate-13, sorbitan stearate, and combinations thereof;
c. an emulsifier selected from the group consisting of sorbitan olivate, ceterayl olivate, and combinations thereof;
d. glycerin; and
e. 3-(4-farnesyloxyphenyl)-propionic acid.
